(19) United States
(12) Patent Application Publication
(10) Pub. No.: US 2016/0157928 A1
(43) Pub. Date: Jun. 9, 2016

(54) VESSEL SEALING AND CUTTING DEVICES, METHODS AND SYSTEMS

(71) Applicant: ASYMMETRIC MEDICAL LTD., Kfar Mordechai (IL)

(72) Inventors: Moshe ESHKOL, Harutzim (IL); Ori WEISBERG, Kfar Mordechai (IL)

(21) Appl. No.: 14/905,833

(22) PCT Filed: Jul. 17, 2014
(86) PCT No.: PCT/IL2014/050646

§ 371 Date: Jan. 18, 2016

Related U.S. Application Data
(60) Provisional application No. 61/847,090, filed on Jul. 17, 2013.

(51) Int. Cl. A61B 18/14; G02B 6/28; G02B 6/14; A61B 17/28; G02B 6/26

(52) U.S. Cl. CPC A61B 18/1445; A61B 17/28; G02B 6/262; G02B 6/14; G02B 6/2852; A61B 2018/00345

(57) ABSTRACT

A vessel sealing tip for surgical forceps is provided, which allows both sealing a vessel section and cutting therethrough without extracting the tip out of the body or exchanging the tip. Either a single action yields the sealing and the cutting or two or more tip actions may be carried out sequentially to perform the sealing and cutting operations. In addition, the tip may be used for cutting through tissue. Embodiments of the tip may utilize any energy source, in particular optical laser energy but also RF or ultrasound energy. The different effects (sealing, cutting) may be achieved by varying the emitted energy spatially, by manipulating the vessel prior or during energy delivery, by changing a configuration of the tip during operation and by combining tensile forces or ablation at appropriate locations of the vessel.

### BACKGROUND OF THE INVENTION

1. Technical Field

The present invention relates to the field of surgery, and more particularly, to vessel manipulation.

2. Discussion of Related Art

Vessel manipulation is a commonly encountered challenge, especially in minimally invasive procedures. The variety of encountered vessels and the need to manipulate vessels without causing additional damage and bleeding require time and skill which may challenge procedure success and place a significant obstacle to the further development of such procedures.

### SUMMARY OF THE INVENTION

One aspect of the present invention provides a vessel sealing tip for surgical forceps, the vessel sealing tip comprising at least one energy delivering element arranged to deliver, upon actuation, energy to a vessel to yield a vessel welding effect in a specified sealing section of the vessel and to cut the vessel within the specified sealing section.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A-1C are high level schematic illustrations of a vessel sealing tip for surgical forceps according to some embodiments of the invention.

FIG. 1D is a high level schematic illustration of a fiber cross section according to some embodiments of the invention.

FIGS. 2A, 2B and 2C are high level schematic illustrations of a vessel sealing tip for surgical forceps having focusing elements, according to some embodiments of the invention.

FIGS. 3A and 3B are high level schematic illustrations of a vessel sealing tip for surgical forceps having vessel piercing elements, according to some embodiments of the invention.

FIGS. 4A and 4B are high level schematic illustrations of a vessel sealing tip for surgical forceps having transversely expanding elements, according to some embodiments of the invention.

FIG. 5 is a high level schematic illustration of a vessel sealing tip for surgical forceps enabling extension of the vessel sealing region, according to some embodiments of the invention.

FIG. 6 is a high level schematic illustration of a vessel sealing tip for surgical forceps with variable intensity treatment, according to some embodiments of the invention.

FIGS. 7A and 7B schematically illustrate surface designs that influence fiber bending, according to some embodiments of the invention.

FIGS. 8A-8C schematically illustrate fiber bending profiles, according to some embodiments of the invention.

FIG. 9 is a high level schematic flowchart illustrating a vessel sealing method, according to some embodiments of the invention.

### DETAILED DESCRIPTION OF THE INVENTION

Prior to the detailed description being set forth, it may be helpful to set forth definitions of certain terms that will be used hereinafter.

The term “tissue” as used herein in this application refers to any bodily tissue, including vessels as defined below and any other type of tissue such as connective tissue, muscle tissue, nervous tissue, specific organs, fatty tissue, epithelial tissue and any combination thereof. The term “vessel” as used herein in this application refers to any bodily vessel, duct or tract. For example, the term “vessel” may refer to a blood vessel, a bile duct, an urinary tract or any other bodily vessel, duct or tract.

The terms “energy” or “treatment energy” as used herein in this application refer to any type of energy which is usable for treating or affecting vessels, for example electromagnetic energy in any form (e.g., optical energy, laser energy in any effective bandwidth, radiofrequency radiation—RF etc.), electrical or magnetic energy (e.g., electric currents or magnetic fields), ultrasonic radiation etc.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Embodiments of the invention provide a vessel sealing tip for surgical forceps which allows both sealing a vessel section and cutting therethrough without extracting the tip out of the body or exchanging the tip. Either a single action yields the sealing and the cutting, or two or more tip actions may be carried out sequentially to perform the sealing and cutting operations. In addition, the tip may be used for cutting through tissue. Embodiments of the tip may utilize any energy source, in particular optical laser energy but also RF or ultrasound energy. The different effects (sealing, cutting) may be achieved by varying the emitted energy spatially, by manipulating the vessel prior or during energy delivery, by changing a configuration of the tip during operation and by combining tensile forces or ablation at appropriate locations of the vessel. In certain embodiments, the vessel sealing tip may be used for general and robotic surgery. The disclosed devices may be used to achieve various tissue effects, e.g., coagulation, welding, sealing, cutting, ablation and combination thereof.

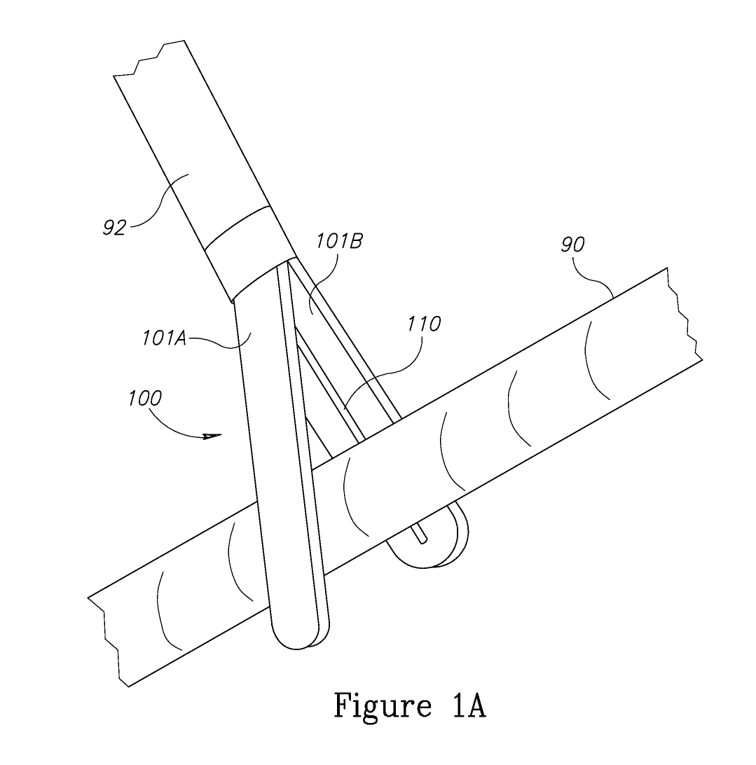

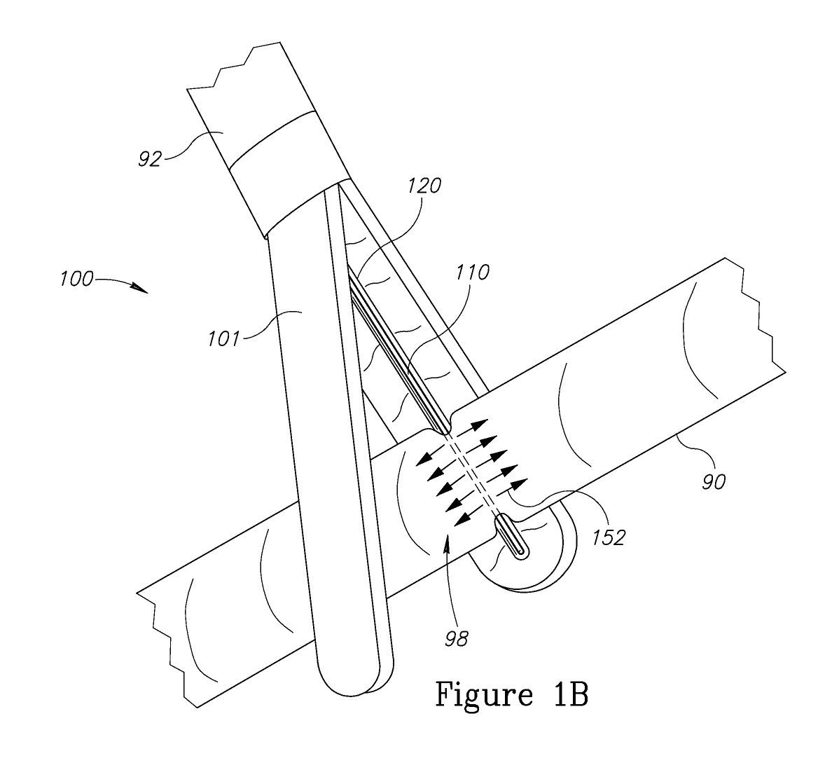

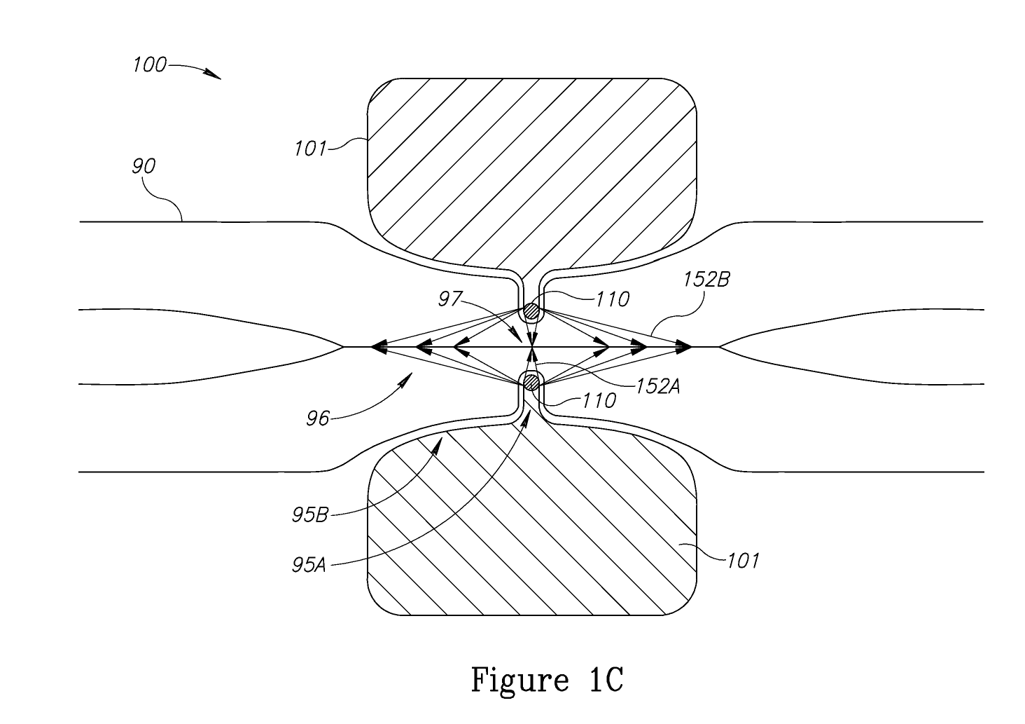

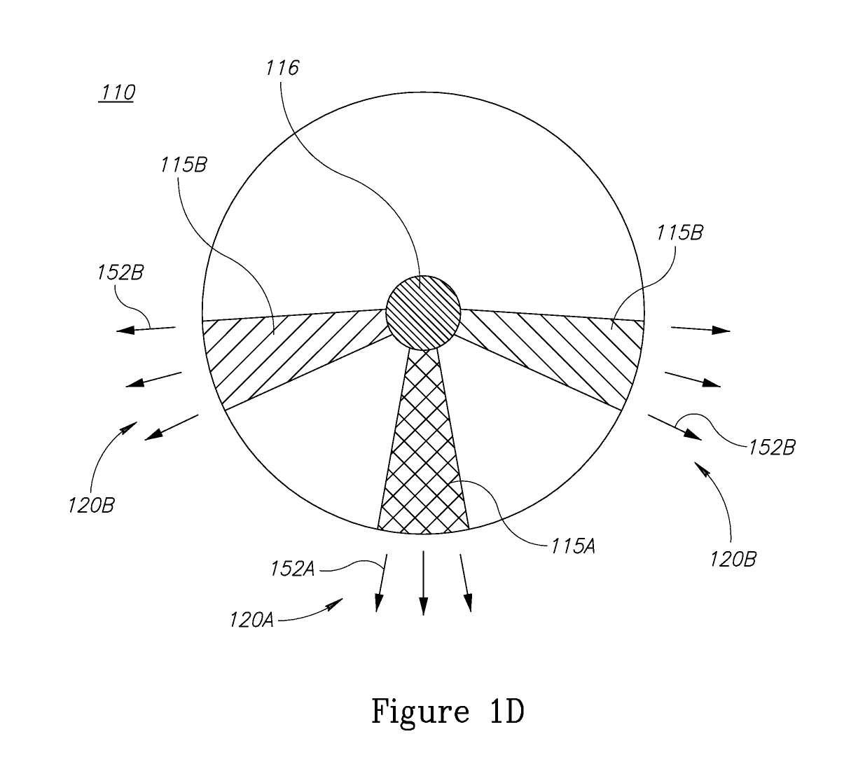

FIGS. 1A-1C are high level schematic illustrations of a vessel sealing tip 100 for surgical forceps 92 according to some embodiments of the invention.

Vessel sealing tip 100 may comprise an energy delivery element 110 such as at least one optical element 110 arranged to deliver, upon actuation, electromagnetic radiation 152 to a vessel 90 to cut vessel 90 at a cutting region 98 (FIG. 1B) or to yield a vessel welding effect in a specified sealing section 96 of vessel 90 (FIG. 1C), and to cut vessel 90 at a cutting location 97 within specified sealing section 96. For example, at least one optical element 110 may comprise at least one optical fiber 110 arranged to deliver electromagnetic radiation such as laser energy. In case of cutting region 98, radiated energy 152 may also seal edges of the cut vessel during the cutting.

Energy delivery element 110 may be attached to any one of two jaws 101 (101A, 101B) of forceps tip 100, or may also be a free element, at least on a part of the length thereof (see below).

In cases of energy delivery element 110 being an optical fiber, fiber 110 may emit radiation 152 (FIG. 1B) that yields a vessel sealing effect and radiation 152 that yields a vessel cutting effect. Radiation characteristics may be temporally varied in a controlled manner or may be designed in advance with respect to one or more vessel types. Radiation 152 may further be used to ablate the vessel wall prior to sealing and/or cutting vessel 110. Radiation 152 may further be used to cut tissue during the advancement of tip 100; for example, fiber 110 may continue beyond the illustrated extension to the very tip of either jaw 101, to their external sides or may extend beyond tip 100 itself (e.g., form a loop ahead of tip 100).

FIG. 1D is a high level schematic illustration of a fiber cross section according to some embodiments of the invention. Fiber 110 may be arranged to emit at least two radiation types 152A, 152B at at least two corresponding zones (120A, 120B, FIGS. 1C, 1D) of fiber 110. The radiation types may differ in at least one of their intensity, spectral range, spatial and/or temporal patterns. Emission zones 120A, 120B may comprise, in cross section, different corresponding fiber sectors 115A, 115B made of different cladding materials or having different refractive indices. In embodiments, emission zones 120A, 120B may have different cross-sections and thus different spatial energy density profiles. In certain embodiments, fiber 110 may comprise a solid core optical fiber (having core 116), a hollow fiber or a photonic crystal fiber (such as a holey fiber, a Bragg fiber or any other micro-structured fiber). In certain embodiments, fiber 110 may comprise a metallic waveguide.

The different fiber sections may be differently micro-structured or have a different spatial arrangement of core 116 and cladding (e.g. core 116 may be asymmetrically positioned within the cladding). Fiber 110 may be single-mode or multi-mode. Beam polarization may also be used to differentiate radiation types 152A, 152B and control the emitted energy density spatial distribution.

In certain embodiments, at least one jaw 101 of the forceps may comprise at least one protrusion 95A (FIG. 1C) arranged to constrict vessel 90 prior to the actuation of energy delivery element 110 (such as at least one optical element 110, a RF source, an ultrasound source etc.). Protrusion 95A protrudes from a surface 95B of jaw 101 and constricts vessel 90 at the region of energy deliver to reduce the local thickness of vessel 90 and to provide more spatial variability in possible energy delivery directions. Energy delivery element 110 may be positioned fully or partially within protrusion 95A; for example, at least one optical element 110 may be set within at least one protrusion 95A.

Certain embodiments of the invention comprise a tip 100 with at least two jaws 101 for surgical forceps 92. At least one of jaws 101 may comprise at least one protrusion 95A positioned to contact tissue held by tip 100 and deliver both pressure and external energy to the tissue. The pressure may be a tip holding force (the force applied to the forceps and thereby transferred to the tip's jaws), concentrated by at least one protrusion 95A. The external energy may be any of electromagnetic (e.g., optical, RF), electrical and ultrasound energy, or a combination thereof. At least one protrusion 95A may comprise one or more thin element that concentrates applied forces onto a small section of vessel 90. At least one protrusion 95A may comprise an abrasive or an ablative element that reduces vessel wall thickness or even cuts the vessel, in addition to constricting the vessel.

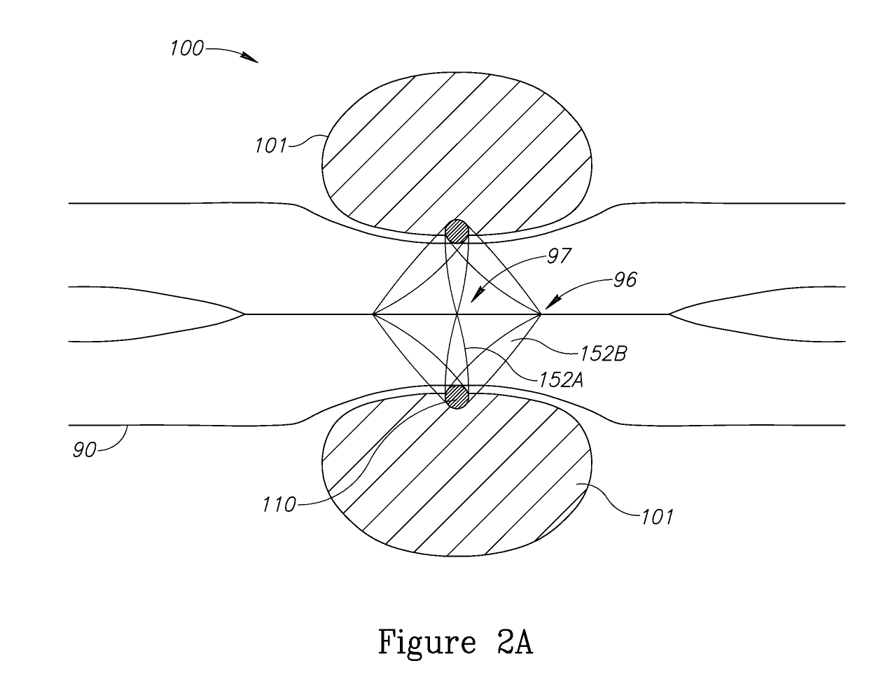

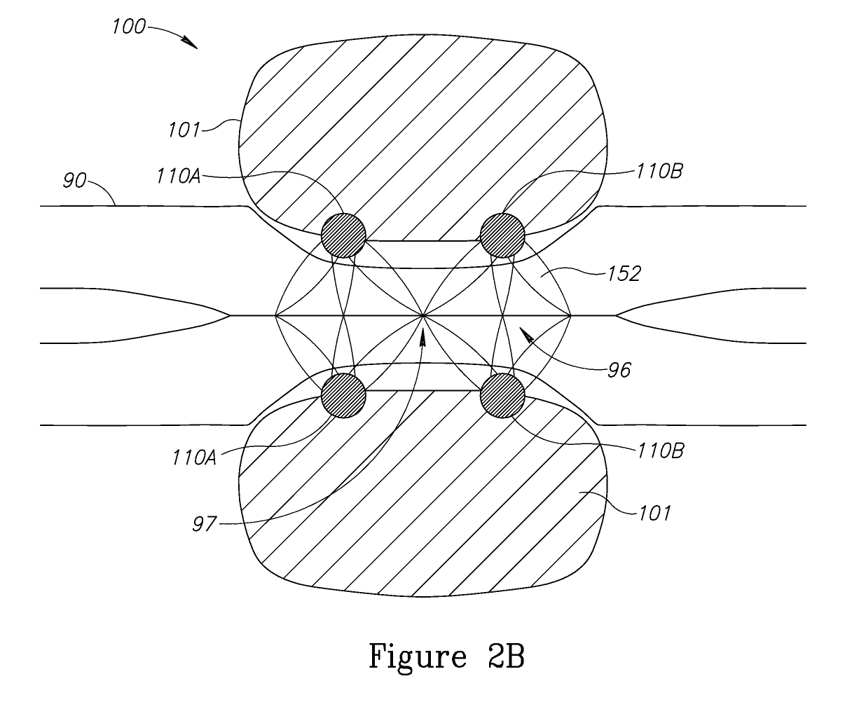

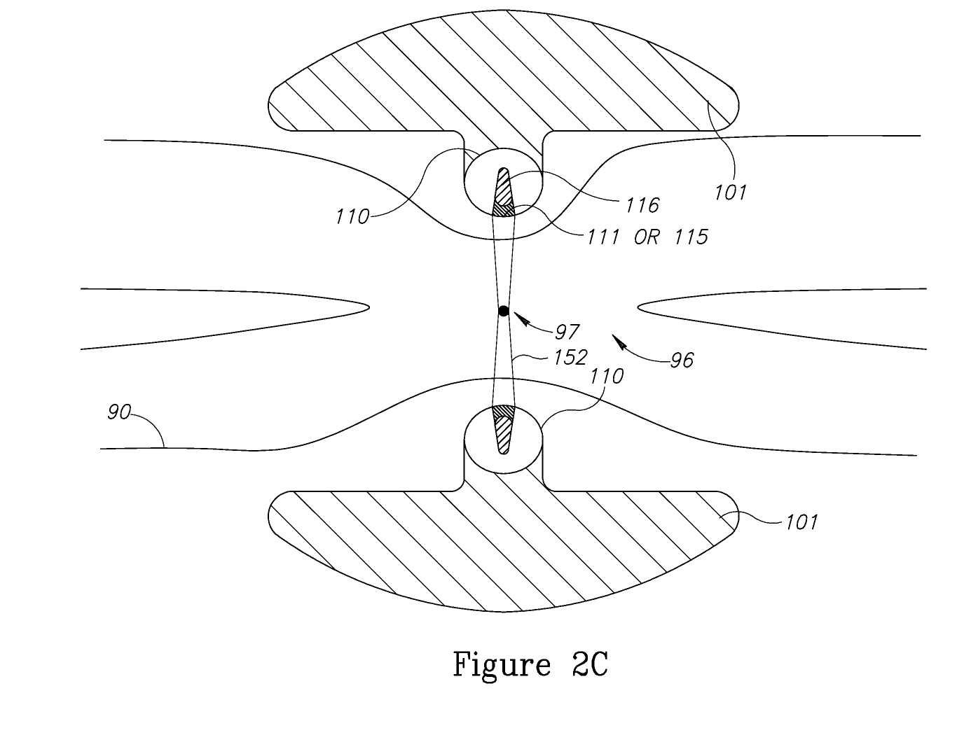

FIGS. 2A, 2B and 2C are high level schematic illustrations of vessel sealing tip 100 for surgical forceps 92 having focusing elements 111, according to some embodiments of the invention. Energy delivery elements 110 may comprise focusing elements 111 (FIG. 2C) arranged to focus any type of delivered energy (e.g., optical energy, RF, ultrasound, electrical energy, etc.). For example, optical elements 110 may comprise at least one focusing element 111, such as a lens 111 or a sector 115 of the cladding arranged to focus emitted radiation 152. In the non-limiting example illustrated in FIG. 2C, a combination of asymmetric core 116 and focusing element 111 may be arranged to yield the welding and/or cutting of vessel 90 (depending on the delivered radiation and tip manipulation). The focusing of the emitted radiation may be in a cross-sectional plane of optical element 110. In certain embodiments, focusing elements 111 may focus different types of radiation 152A, 152B at different regions of vessel 90, e.g., radiation 152B may be focused to produce a sealing effect at sealing region 96, and radiation 152A may be focused to cut vessel 90 at cutting region 97. Focusing elements 111 may be embedded in or attached to forceps jaws 101. Focusing elements 111 may be multiply associated with at least one of jaws 101, as illustrated in FIG. 2B. The focusing elements may be embedded in multiple fibers 110A, 110B and be arranged to jointly apply the sealing and cutting to regions 96, 97 of vessel 90 respectively. Particularly, at least two focusing elements 111 may be positioned on each jaw 101 and arranged to yield a specified extension of specified sealing section 96, which is broader than sealing section 96 produced by a single focusing element 111 or solely by optical element 110.

In certain embodiments, energy delivery element 110 may be arranged to reduce a vessel wall thickness prior to the welding. For example, optical element 110 may operate in an ablative mode to reduce vessel wall thickness prior of holding vessel 90 sealing it and cutting through vessel 90. The reduction of wall thickness allows energy to be delivered to the internal walls of vessel 90 without causing thermal damage to the external wall of vessel 90. Furthermore, reducing the wall thickness may reduce the wall resistance to mechanical pressure and thus allow a more effective application of pressure to vessel 90, e.g., by protrusions 95A (FIG. 1C), to create a more effective gripping of vessel 90 by forceps 92 and a better sealing effect.

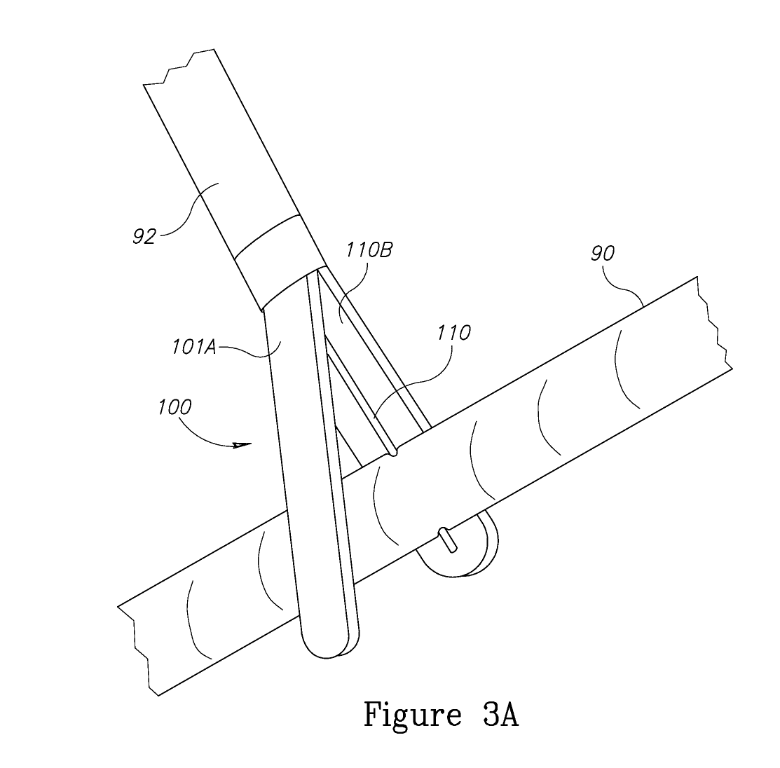

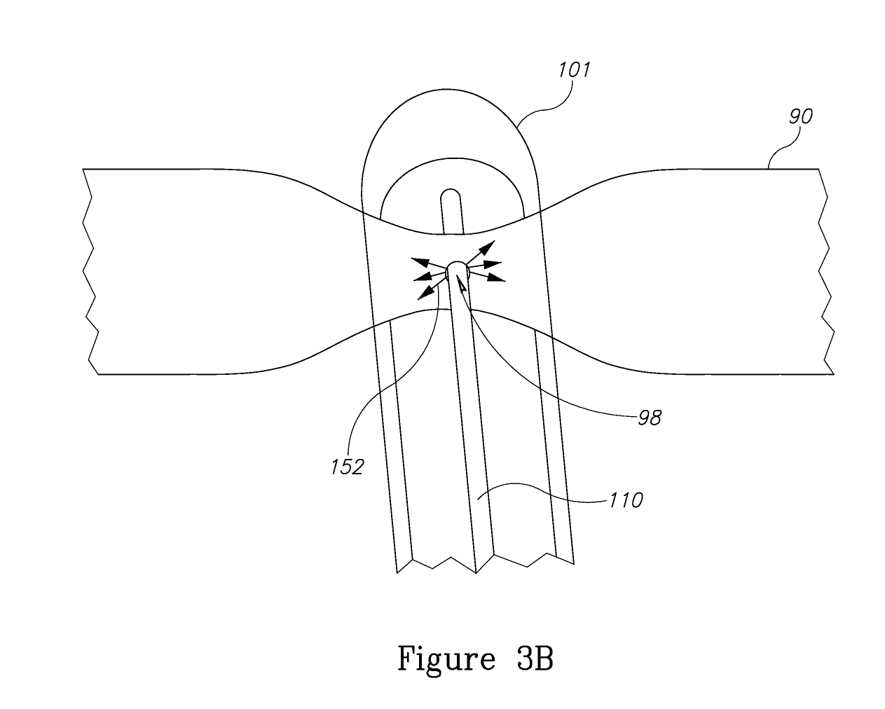

FIGS. 3A and 3B are high level schematic illustrations of vessel sealing tip 100 for surgical forceps 92 having vessel piercing elements 110, according to some embodiments of the invention. Energy delivery element 110 may comprise at least one optical element 110 comprising at least one optical fiber 110 arranged to penetrate a lumen of vessel 90, piercing thereby a hole 98 in vessel 90, prior to the actuation thereof and emission of energy 152 from fiber 110. In certain embodiments, penetrating the vessel lumen enables more efficient sealing and/or cutting of vessel 90. Delivering energy from the interior of vessel 90 allows treating its inner layers directly, without having to apply high pressure on the vessel wall in order to flatten vessel 90. In certain embodiments, penetrating and flattening may be applied simultaneously or sequentially to reciprocally enhance the sealing effect.

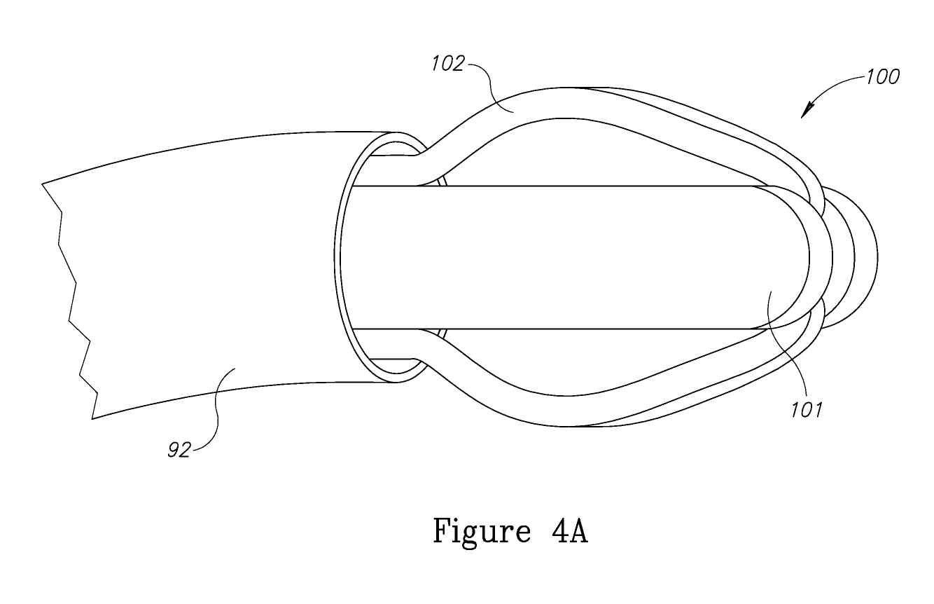

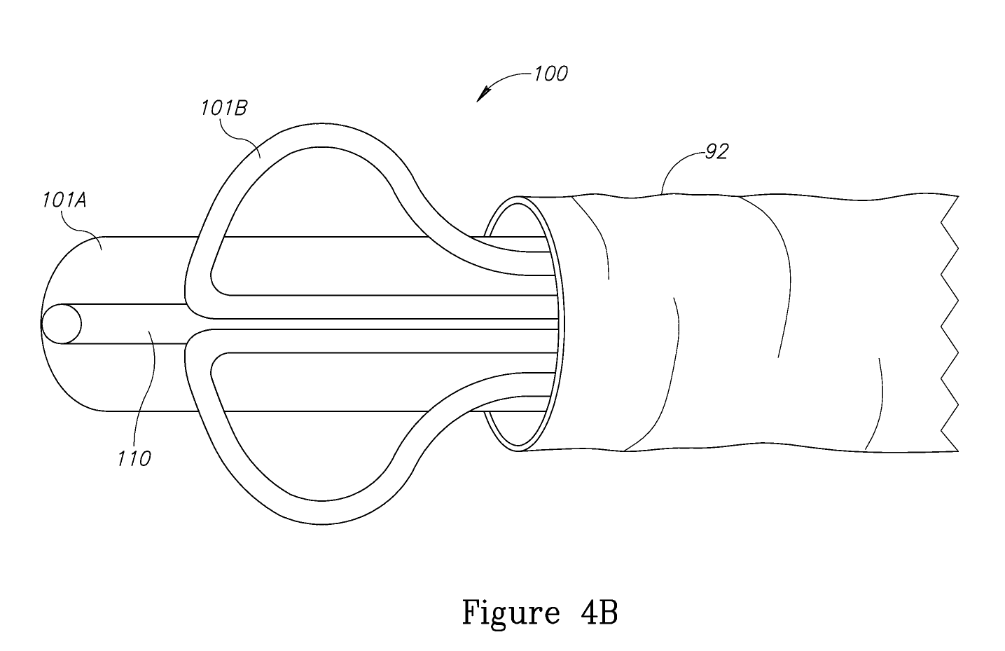

FIGS. 4A and 4B are high level schematic illustrations of vessel sealing tip 100 for surgical forceps 92 having transversely expanding elements 102, according to some embodiments of the invention. At least one jaw 101 of tip 100 may comprise transversely expandable element 102 arranged to yield a specified extension of specified sealing section 96. Mechanically pressing a wider section of vessel 90 increases the potential sealing section of vessel 90 and hence may improve sealing and vessel manipulation conditions. Transversely expanding elements 102 may be retracted in a tool delivery channel and expand only in situ, upon using tip 100. In certain embodiments, transversely expanding elements 102 are controllably expandable, e.g., by a user of forceps 92 or responsive to applied pressure on respective jaw 101 or sensed resistance of vessel 90. In certain embodiments, transversely expanding elements 102 may mechanically extend specified sealing section 96 during the welding. The welding (e.g., by radiation 152B aimed at sealing section 96) may be combined with transverse forces applied to vessel 90 at the sealing section and aimed to expand the treated section. A reduced resistance of vessel 90 due to the energy radiation and/or the mechanical extending may thus be exploited to expand the sealing section. In certain embodiments (FIG. 4B), at least one of jaws 101 may be designed as a transversely expanding element. For example, one jaw 101A may be a regular jaw and the other jaw 101B may be expandable to broaden the sealing region of vessel 90. Expandable jaw 101B may be made of two or more parts and tip 100 may comprise means to separate the parts to further enhance the stretching effect on vessel 90.

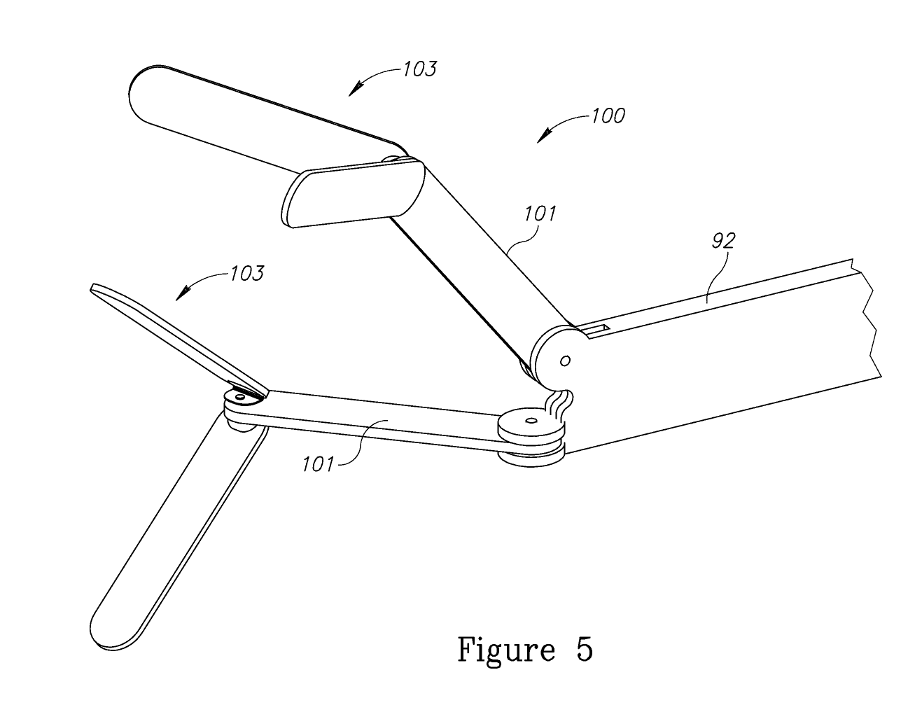

FIG. 5 is a high level schematic illustration of vessel sealing tip 100 for surgical forceps 92 enabling extension of the vessel sealing region, according to some embodiments of the invention. In certain embodiments, one or both jaws 101 may be hingedly attached to forceps 92 and may be controllably pivotally movable to stretch vessel 90, e.g., during sealing or cutting thereof. One or both jaws 101 may comprise a forceps element 103 arranged to hold and/or pull a respective vessel section to generate the stretching effect of the vessel, which expands the sealing region. Jaws 101 may be moveable along different spatial directions, to yield an additional twist of vessel 90, selected to further enhance the stretching of the sealing region. Any of the above mentioned movements and actions may be combined with energy delivery to enhance the sealing and/or cutting effect. Accordingly, control of any of these movements may be carried out by a user or responsive to sensed forces in tip 100. Jaws 101 may by controlled by mechanical compliance to exerted forces.

In certain embodiments, vessel sealing tip 100 for surgical forceps 92 may comprise at least one transversely expandable element 110 or 103 arranged to yield a specified extension of a specified section of vessel 90 and energy delivery element 110 arranged to deliver external energy, upon actuation, to vessel 90 to yield a vessel welding effect in a specified sealing section of vessel 90 and to cut vessel 90 within the specified sealing section. The external energy may be at least one of optical, electrical and ultrasound energy. Tip 100 may thus open up and create a seal larger than half width of tool (e.g., tip 100) or just separate regions of cut and seal. The specified sealing section may be mechanically extended during the welding with or without additional energy delivery. In embodiments, tip 100 may comprise two transversely expandable elements 103, each arranged to yield a specified extension of the specified section of vessel 90 in a different plane.

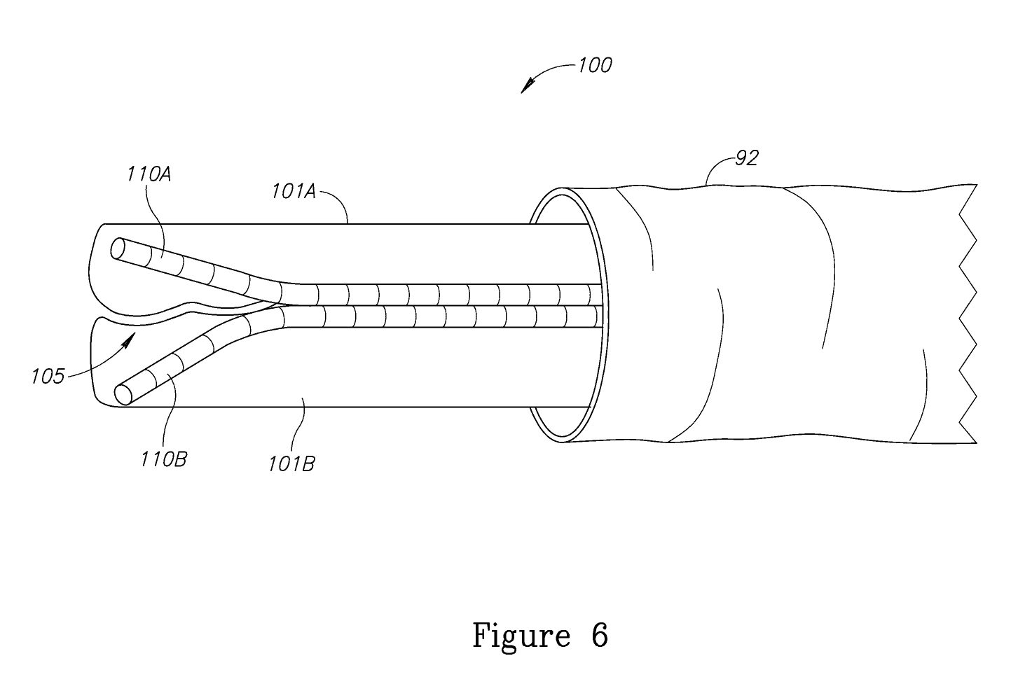

FIG. 6 is a high level schematic illustration of vessel sealing tip 100 for surgical forceps 92 with variable intensity treatment, according to some embodiments of the invention. In certain embodiments, each jaw 101A, 101B of the forceps may comprise at least one optical fiber 110A, 110B respectively, positioned at a distance from an edge 105 of the respective jaw, wherein the distance may vary along the jaws. For example, the varying distance with respect to treating edge 105 of the jaws may diminish from a tip to a base of jaws 101A, 101B to yield the welding effect at the jaw tip and perform the cutting between the tip and the base of the jaw. In certain embodiments, the sealing and cutting effects are thus spatially differentiated along the jaws instead or in addition to the spatial differentiation across the jaws illustrated previously (cf., e.g., FIG. 2A). In certain embodiments, one or more fibers 110A, 110B may have longitudinally varying characteristics that generate radiation 152 of different characteristics along the fiber. For example, radiation 152 designed for sealing may be applied at the jaw tips where the distance to vessel 90 is also the largest, and radiation 152 designed for cutting may be applied at the jaw bases where the distance to vessel 90 is also the smallest. Hence jaws 101 and energy delivery elements 110 may be designed to jointly differentiate welding and cutting effects.

In certain embodiments, vessel sealing tip 100 may be constructed from non-metallic materials to allow use of tip 100 simultaneously with MRI imaging. For example, tip 100 may be made of plastic and energy may be delivered via optical fibers.

In certain embodiments, vessel sealing tip 100 may comprise at least one wave guide (not shown) arranged to deliver, upon actuation, electromagnetic radiation to the vessel to yield a vessel welding effect in a specified sealing section of the vessel and to cut the vessel within the specified sealing section. In certain embodiments, at least one jaw of the forceps may comprise at least one protrusion arranged to constrict the vessel prior to the actuation of the at least one wave guide.

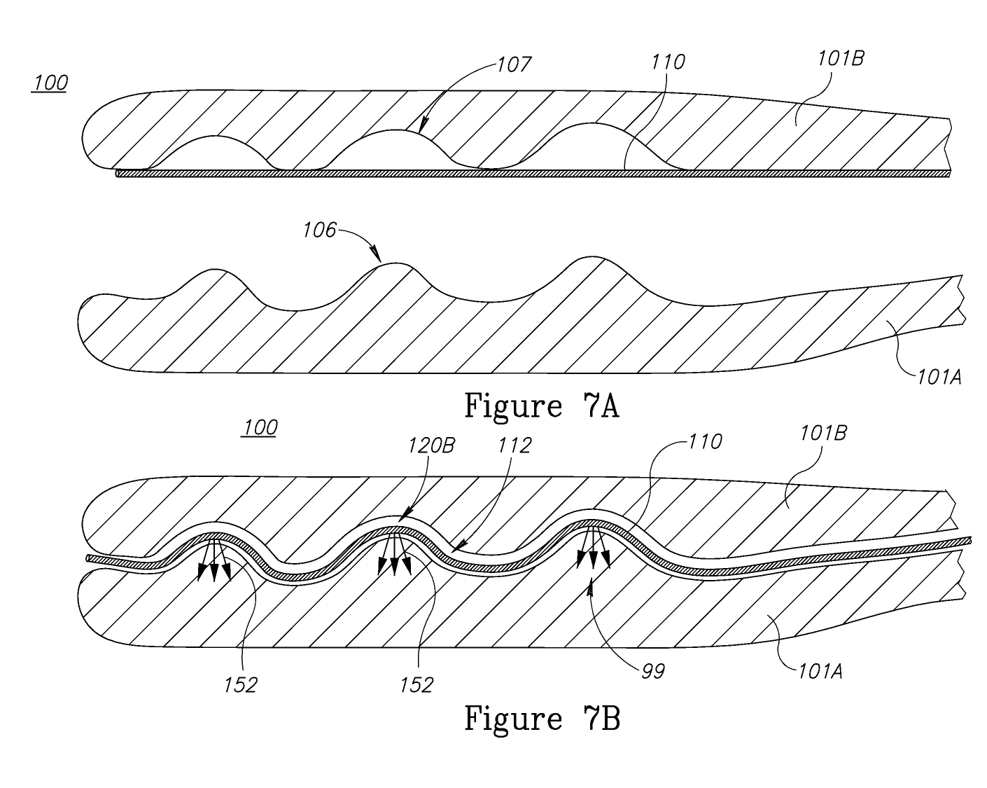

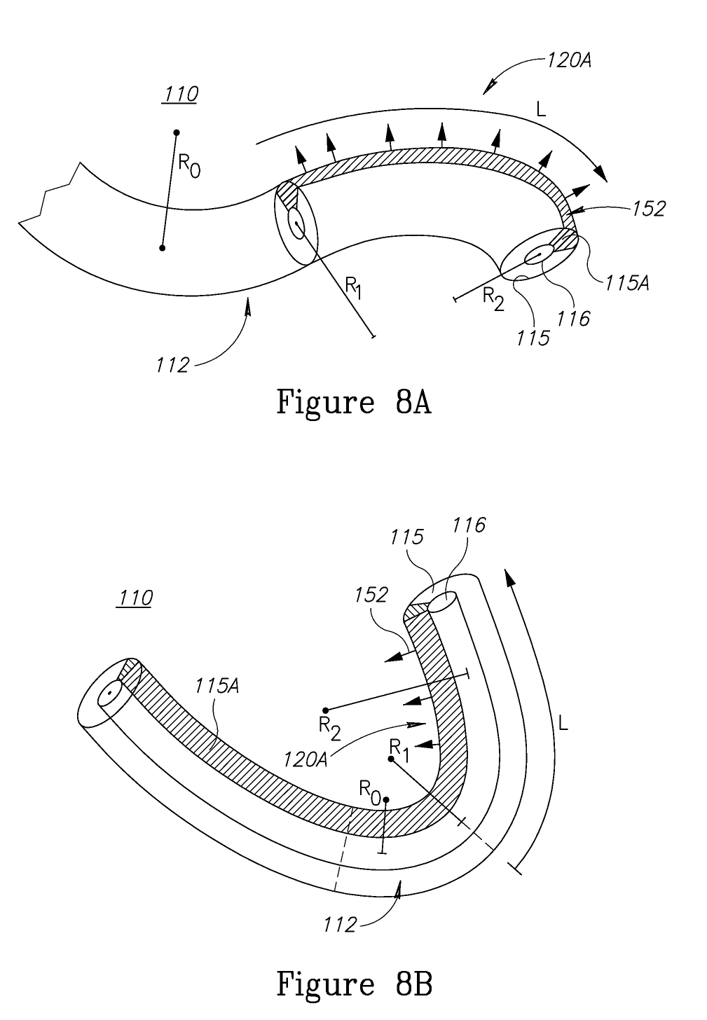

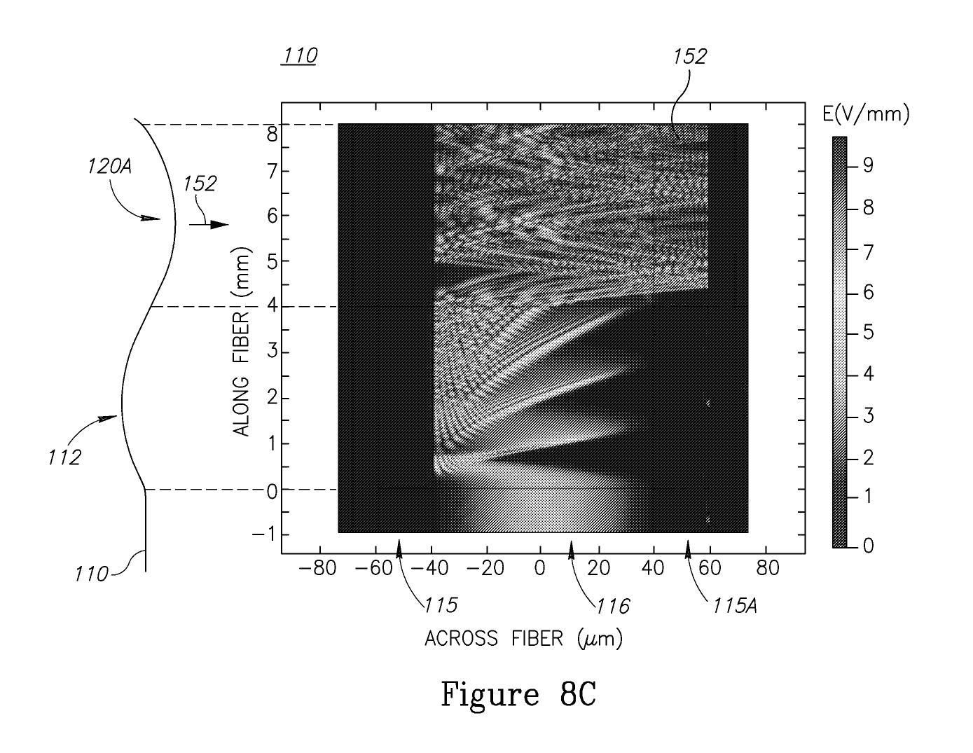

FIGS. 7A and 7B schematically illustrate surface designs that influence fiber bending, according to some embodiments of the invention. FIGS. 7A and 7B illustrate, respectively, an open, non-emitting, position and an active position of tweezers-like device 100, e.g., vessel sealing tip 100. Fiber 110 is integrated within tweezers device 100 in a way that causes bending of fiber 110 upon handling tissue with device 100 and radiation emission from the bended regions 120B which enhances treatment of the handled tissue. For example, fiber 110 may be associated with one arm 101B of tweezers device 100 and fiber bending may occur upon pressing the fiber against a second arm 101A of tweezers device 100. Any of the tweezers' arms may comprise protrusions 106 and/or corresponding recesses 107 to enhance fiber bending upon handling tissue by tweezers 100 and thereby yield treatment at regions 99 which may be sealing regions 96 and/or cutting regions 97, 98. Protrusions 106 and/or corresponding recesses 107 may be further designed to define pre-bend regions 112 as explained below (FIGS. 8A-8C). Tweezers-like device 100 may comprises surface features designed to control the bending of optical fiber 110 upon tissue contact. For example, tweezers device 100 may have multiple fibers 110 which may have differing emission characteristics, e.g., configured to apply different effects to the treated tissue, and/or tweezers device 100 may have multiple types of protrusions 106 and recesses 107 having different curvatures and hence determining different types of emitted radiation and respective effects on the tissue. Tweezers device 100 hence allows mechanical handling while using laser for welding and/or cutting tissue. The emission may be dependent on the extent of the force applied by the physician through the extent of resulting bending of fiber(s) 110. The closer arms 101A, 101B are pressed together, the larger becomes the fiber bending and the emitted radiation.

FIGS. 8A-8C schematically illustrate fiber bending profiles, according to some embodiments of the invention. FIGS. 8A and 8B schematically illustrate two types of fiber bending and corresponding emission zones 120A, while FIG. 8C illustrates simulation results that exemplify the configuration of pre-bend region 112. Both FIGS. 8A and 8B illustrate pre-bend region 112 and FIG. 8C illustrates the operation of pre-bend region 112.

FIG. 8A schematically illustrates fiber 110 having emitting zone 115A at emission region 120A, emitting radiation 152 outwardly from convex emission region 120A. Emission region 120A is characterized by a decreasing radius of curvature R₂<R₁ along its length L (the arrow marks the direction of radiation propagation) and generally dR/dL<0 along emission region 120A. Emission region 120A is preceded by pre-bend region 112 having an opposite curvature (R₀) and configured to enhance emission from emitting zone 115A upon the bending of emission region 120A. Protrusions 106 and/or recesses 105 in supportive structures may be used to define the changing of the radius of curvature mechanically, or the radius may be changed by the practicing physician manually or electronically.

FIG. 8B schematically illustrates fiber 110 having emitting zone 115A at emission region 120A, emitting radiation 152 inwardly from concave emission region 120A. Emission region 120A is characterized by an increasing radius of curvature R₂>R₁ along its length L (the arrow marks the direction of radiation propagation) and generally dR/dL>0 along emission region 120A. Emission region 120A is preceded by pre-bend region 112 having still stronger curvature R₀<R₁ and configured to enhance emission from emitting zone 115A upon the bending of emission region 120A. Protrusions 106 and/or recesses 105 in supportive structures may be used to define the changing of the radius of curvature mechanically, or the radius may be changed by the practicing physician manually or electronically. Other techniques may be used to yield and use inwards emission by bending the fiber.

FIG. 8C schematically illustrates simulation results that exemplify the functioning of pre-bend region 112 in the configuration illustrated in FIG. 8A. The electromagnetic finite elements simulation illustrates a cross section of fiber 110 along pre-bend region 112 and emission region 120A. The x axis is across fiber 110 (−75 μm to +75 μm), including non-emitting cladding zone 115 (−75 μm to −40 μm), core 116 (−40 μm to +40 μm) and emitting cladding zone 115A (+40 μm to +75 μm). It is noted that the simulated fiber is actually bent as illustrate to the left of the simulation results, while simulation results are shown for convenience along a seemingly straight fiber, using a conformal mapping technique. The y axis is along fiber 110 (−1 mm to +8 mm), including a straight (not bended) fiber region (−1 mm to 0 mm), pre-bend region 112 (0 mm to 4 mm) and emitting region (4 mm to 8 mm). Radiation intensity (simulated norm strength of the electric field) is indicated by gray levels from zero (black) to ca. 6000 (white) Volts/m and beyond (larger values are scarcely represented in the illustration and are seen as black streaks at the very edge of the intensity maxima at ca. +0.5 mm and 2 mm, left to the center of the core). While intensity distribution along central non-bent region is Gaussian about the center of core 116, pre-bend region 112 exhibits higher propagation modes and energy concentration at the left, convex side of the bending. No emission is observed from the fiber due to the high energy barrier in the direction opposing the emission zone. The pre-bend radius R₀ may be large or small, without resulting in emission. Upon bending the fiber to the other direction at emitting region 120A, the eccentrically distributed energy is emitted through respective cladding zone 115A. Bending radii R₀, R₁, R₂ and the refractive indices of core 116, non-emitting cladding 115 and emitting cladding zone 115A are configured according to specified required rending configurations and performance requirements. For the configuration illustrated in FIG. 8B, similar eccentric concentration of energy occurs along pre-bend region 112, but energy is released through emission zone 115A upon increasing the radius of curvature and utilizing the change in energy distribution that is illustrated in FIG. 8C in the regions between 4-4.5 mm and shows a tendency of the energy distribution to move to the right side of the fiber upon increasing the curvature radius. Clearly, the refractive indices are configured to enhance this effect.

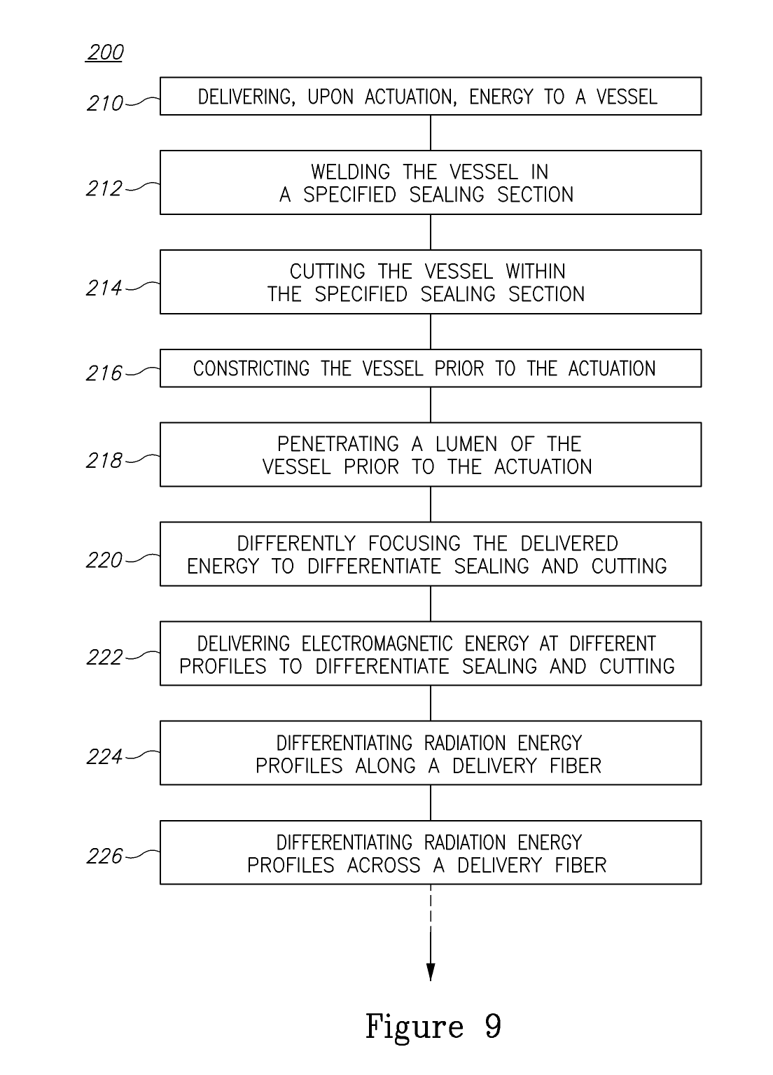

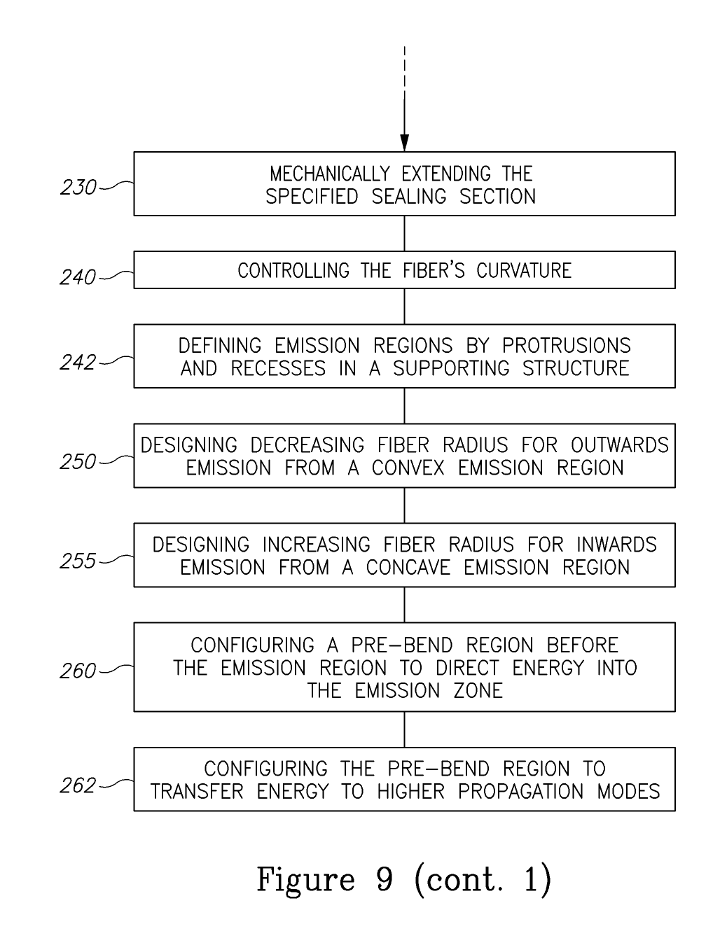

FIG. 9 is a high level schematic flowchart illustrating a vessel sealing method 200, according to some embodiments of the invention. Method 200 may be used to achieve different tissue effects, ranging from welding through sealing to cutting, ablation and any combination of these and other effects (e.g., coagulation).

Vessel sealing method 200 comprises delivering, upon actuation, energy to a vessel (stage 210) to yield a vessel welding effect in a specified sealing section of the vessel and to cut the vessel within the specified sealing section. Method 200 may comprise welding the vessel in a specified sealing section (stage 212) and cutting the vessel within the specified sealing section (stage 214). In certain embodiments, the welding and the cutting may be carried out by a single actuation. The delivered energy may comprise at least one of optical, electrical and ultrasound energy

For example, the delivered energy may be electromagnetic radiation and method 200 may further comprise creating the welding and cutting by differently focusing the delivered electromagnetic radiation on the specified sealing section and on the cutting location, respectively, to differentiate sealing and cutting (stage 220).

In another example, the delivered energy may be electromagnetic radiation method 200 may further comprise using at least one optical fiber arranged to emit the electromagnetic radiation at at least two radiation profiles, one corresponding to welding 212 and another corresponding to cutting 214 the vessel. Generally, certain embodiments may comprise delivering electromagnetic energy at different profiles to differentiate sealing and cutting (stage 222). For example, radiation energy profiles may be differentiated along a delivery fiber (stage 224), across a delivery fiber (stage 226) or by a combination thereof and in respect to the positioning of the delivery fibers in jaws of a forceps tip arranged to perform method 200.

In certain embodiments, method 200 may further comprise constricting the vessel prior to the actuation (stage 216). The constriction may be arranged to yield more effective sealing and/or cutting by reducing the vessel diameter and increasing the usable spatial variability of energy delivery.

In certain embodiments, method 200 may further comprise penetrating a lumen of the vessel prior to the actuation (stage 218). Penetrating the vessel enables sealing the vessel from within and thereby applying the delivered energy efficiently and in a controllable manner to seal and cut the vessel.

In certain embodiments, method 200 may further comprise mechanically extending the specified sealing section (stage 230). The extending may be carried out prior, during or after sealing the vessel to broaden the sealing section to allow more effective cutting and healing of the cutting location.

Method 200 may further comprise controlling the fiber's curvature (stage 240), for example by defining emission regions by protrusions and recesses in a supporting structure (stage 242) such as forceps, tweezers or any other structure.

Method 200 may comprise configuring emission from an optical fiber by arranging at least one specified region in the optical fiber to emit transferred electromagnetic radiation from a core through a cladding of the optical fiber upon bending of the optical fiber at the at least one specified region beyond a specified bending threshold.

Method 200 may comprise designing decreasing fiber radius for outwards emission from a convex emission region (stage 250). Alternatively or additionally, method 200 may comprise designing increasing fiber radius for inwards emission from a concave emission region (stage 255).

Protrusions and/or recesses in the supportive structure may be used to define the changing of the radius of curvature mechanically.

In certain embodiments, method 200 may further comprise configuring a pre-bend region before the emission region to direct energy into the emission zone (stage 260) and/or configuring the pre-bend region to transfer energy to higher propagation modes (stage 262).

In certain embodiments, vessel sealing tip 100 for surgical forceps 92 may be configured to be applied for any of the following treatments: Sealing blood vessels, arteries, veins; Sealing biliary ducts; Sealing urinary tract; Sealing reproductive tract; Sealing airways; Sealing in the GI tract; Sealing the dura; Treating septums (nasal, atrial, etc.); Sealing organs such as lung, liver, spleen, heart, stomach, pancreas, uterus, bladder, kidney etc. While the above description mainly referred to treating vessels 90, tip 100 for surgical forceps 92 may be configured for treating any other type of tissue, as well as to carry out further surgical tasks, such as cutting or ablating tissue.

In a non-limiting example, vessel sealing tip 100 may be configured to apply pressures in the at least a part of the range 20-400 PSI. The outer diameter of fiber(s) 110 may be between 0.05-2 mm and fiber(s) 110 may be arranged to deliver power levels between e.g. 1 W-100 W. Tip 100 may be configured to have a jaw 101 length between 2-50 mm, a jaw 101 width between 0.5-10 mm, and a ridge width of at least one protrusion 95A between 0.1-5 mm. The dimensions of jaws 101 may be configured with respect to the specific use of tip 100, as illustrated in the examples above. For example, larger tips 100 may be designed to seal larger or stiffer vessels 90.

Table 1 is a non-limiting exemplary overview of possible tip characteristics for various applications of tip 100.

TABLE 1
Tip parameters for various applications

| Vessel type (Sealing | | | Jaw | |
|---|---|---|---|---|
| operation, unless | Anatomical size | Jaw length | width | Working area |
| otherwise indicated) | (mm) | (mm) | (mm) | length (mm) |
| Blood vessels, arteries, | <1 to 10 | 20 and up | 2 to 10 | 17 and up |
| veins | | | | |
| Extremely large blood | up to 25 | 40 to 60 | 2 to 10 | 35 and up |
| vessel, aorta, aneurisms, | | | | |
| etc. | | | | |
| Biliary ducts | 5-10 | 20 and up | 2 to 10 | 8 and up |
| Urinary tract | up to 10 | 20 and up | 2 to 10 | 17 and up |
| Reproductive tract | Fallopian tubes up to | 20 and up | 2 to 10 | 17 and up |
| | 2, general tissue | | | |
| | much more | | | |
| Airways | | 20 and up | 2 to 10 | 17 and up |
| GI tract | | 30 and up | 2 to 10 | 25 and up |
| Dura | | 20 and up | 2 to 10 | 15 and up |
| Septum (nasal, atrial, | | 3 and up | 1 to 10 | 2 and up |
| etc.) | | | | |
| Operating on organs such | | 20 and up | 2 to 10 | 17-70 |
| as lung, liver, spleen, | | | | |
| heart, stomach, pancreas, | | | | |
| uterus, bladder, kidney, | | | | |
| etc. | | | | |
| Neurological operations | | 3 and up | 2 to 10 | 3 and up |
| Kidney operations | | 20 and up | 2 to 10 | 17 and up |

In the above description, an embodiment is an example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A vessel sealing tip for surgical forceps, the vessel sealing tip comprising at least one optical fiber arranged to deliver, upon actuation, electromagnetic radiation to a vessel to yield a vessel welding effect in a specified sealing section of the vessel and optionally to cut the vessel within the specified sealing section.

2. (canceled)

3. The vessel sealing tip of claim 1, wherein at least one jaw of the forceps comprises at least one protrusion arranged to constrict the vessel prior to the actuation of the at least one optical fiber.

4-10. (canceled)

11. The vessel sealing tip of claim 1, wherein the at least one optical fiber is further arranged to reduce a vessel wall thickness prior to the welding.

12. (canceled)

13. The vessel sealing tip of claim 1, further arranged to mechanically extend the specified sealing section during the welding.

14. (canceled)

15. (canceled)

16. The vessel sealing tip of claim 1, constructed from non-metallic materials.

17-23. (canceled)

24. A vessel sealing method comprising delivering, upon actuation, electromagnetic radiation to a vessel to yield a vessel welding effect in a specified sealing section of the vessel and optionally to cut the vessel within the specified sealing section.

25. The vessel sealing method of claim 24, wherein both the welding and the cutting are performed and are carried out by a single actuation.

26. (canceled)

27. (canceled)

28. The vessel sealing method of claim 24, wherein the delivered energy is electromagnetic radiation and the method further comprises using at least one optical fiber arranged to emit the electromagnetic radiation at at least two radiation profiles, one corresponding to welding and another corresponding to cutting the vessel.

29. (canceled)

30. (canceled)

31. The vessel sealing method of claim 24, further comprising constricting the vessel prior to the actuation.

32. (canceled)

33. (canceled)

34. A method of configuring emission from an optical fiber, the method comprising arranging at least one specified region in the optical fiber to emit transferred electromagnetic radiation from a core through a cladding of the optical fiber upon bending of the optical fiber at the at least one specified region beyond a specified bending threshold.

35. The method of claim 34, further comprising designing a decreasing fiber radius for outwards emission from a convex emission region or an increasing fiber radius for inwards emission from a concave emission region.

36. (canceled)

37. The method of claim 34, further comprising configuring a pre-bend region before the emission region to direct energy into the emission region by transferring energy to higher propagation modes.

38. (canceled)

39. The method of claim 34, further comprising configuring at least one protrusions or recess in a fiber supportive structure to define the changing of the fiber radius.

40. An optical fiber comprising a core having a refractive index and a cladding having a refractive index, the optical fiber having at least one specified region of the cladding that is arranged to emit electromagnetic radiation from the core upon bending the optical fiber at the at least one specified region beyond a specified bending threshold.

41. The optical fiber of claim 40, further comprising a decreasing fiber radius for outwards emission from a convex emission region.

42. The optical fiber of claim 40, further comprising an increasing fiber radius for inwards emission from a concave emission region.

43. The optical fiber of claim 40, further comprising a pre-bend region before the emission region, configured to direct energy into the emission region by transferring energy to higher propagation modes.

44. (canceled)

45. The optical fiber of claim 40, further comprising at least one protrusions or recess in a fiber supportive structure, configured to define the changing of the fiber radius.

46. The vessel sealing tip of claim 1, wherein the at least one optical fiber is configured to have at least one specified region that is arranged to emit the electromagnetic radiation from a core of the at least one optical fiber through a cladding thereof upon bending of the at least one optical fiber at the at least one specified region beyond a specified bending threshold.
